(19) United States
(12) Patent Application Publication
(10) Pub. No.: US 2026/0001833 A1
(43) Pub. Date: Jan. 1, 2026

(54) COMPOUNDS, COMPOSITIONS, AND METHODS FOR REDUCING PRODUCTION OF TRIMETHYLAMINE

(71) Applicant: The Cleveland Clinic Foundation, Cleveland, OH (US)

(72) Inventors: Stanley L. Hazen, Cleveland, OH (US); James T. Anderson, Cleveland, OH (US)

(21) Appl. No.: 18/992,077

(22) PCT Filed: Jul. 7, 2023
(86) PCT No.: PCT/US23/69803

§ 371 Date: Jan. 7, 2025

Related U.S. Application Data
(60) Provisional application No. 63/359,640, filed on Jul. 8, 2022.

(51) Int. Cl. C07C 219/06; A61K 31/045; A61K 31/122; A61K 31/145; A61K 31/202; A61K 31/221; A61K 31/225; A61K 31/245; A61K 31/265; A61K 31/575; A61K 31/60; A61K 33/245; A61K 35/00; A61K 35/741; A61K 36/63; A61K 36/68; A61K 36/88; C07C 219/28; C07C 307/02

(52) U.S. Cl. CPC C07C 219/06; A61K 31/045; A61K 31/122; A61K 31/145; A61K 31/202; A61K 31/221; A61K 31/225; A61K 31/245; A61K 31/265; A61K 31/575; A61K 31/60; A61K 33/245; A61K 35/741; A61K 36/63; A61K 36/68; A61K 36/88; C07C 219/28; C07C 307/02; A61K 2035/115

(57) ABSTRACT

Provided herein are substituted quaternary amine salt compounds and compositions thereof for inhibiting production of trimethylamine (TMA), as well as inhibiting the conversion of choline to trimethylamine, and such compounds, and compositions thereof, utilized for treating for example, kidney disease, diabetes and cardiovascular disease, disorders that are associated with inhibiting the conversion of choline to trimethylamine.

### CROSS-REFERENCE TO RELATED APPLICATIONS

This application claims priority to and the benefit of U.S. Provisional Patent Application No. 63/359,640, filed on Jul. 8, 2022, which is incorporated herein by reference in its entirety.

### STATEMENT REGARDING FEDERALLY SPONSORED RESEARCH

This invention was made with government support under HL103866 awarded by the National Institutes of Health. The government has certain rights in the invention.

### FIELD

Provided herein are compounds and compositions that inhibit production of trimethylamine (TMA), and methods of using the compounds, e.g., for inhibiting production of trimethylamine, and for treating disorders associated with conversion of choline to trimethylamine.

### BACKGROUND

TMA and its derivative trimethylamine N-oxide (TMAO) are metabolites linked to disorders such as kidney disease, diabetes mellitus, obesity, trimethylaminuria, and cardiovascular disease (CVD). TMA is produced in the gut by bacteria that are capable of converting substrates, such as choline, to TMA. There is an unmet need for compounds that inhibit the production of TMA by bacteria.

### SUMMARY

In one aspect, disclosed herein are compounds of formula (I):

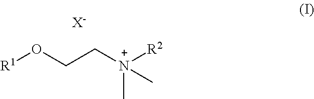

and pharmaceutically acceptable salts thereof, wherein:
R¹ is selected from —C(O)ZR^{a} and —S(O)₂R^{b};
Z is selected from a bond, —O—, and —NH—;
R^{a} is selected from alkyl, alkenyl, alkynyl, aryl, and arylalkyl, each of which is optionally substituted with 1, 2, or 3 substituents independently selected from hydroxy, —COOH, alkyl, alkoxy, halo, haloalkyl, and a group

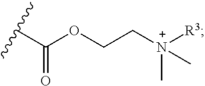

R^{b} is selected from alkyl, alkenyl, alkynyl, aryl, arylalkyl, and —NR^{c}R^{d};
R^{c} and R^{d} are each independently selected from hydrogen, alkyl, alkenyl, alkynyl, aryl, and arylalkyl;
R² and R³ are each independently selected from halomethyl and C₃-C₆ cycloalkyl; and
X⁻ is an anion.

In some embodiments, R¹ is —C(O)ZR^{a}. In some embodiments, R^{a} is C₁-C₆ alkyl, which is unsubstituted or substituted with one —COOH group. In some embodiments, R^{a} is aryl, which is unsubstituted or substituted with one substituent selected from —OH and a group

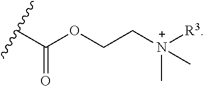

In some embodiments, R^{a} is phenyl substituted with one substituent selected from —OH and a group

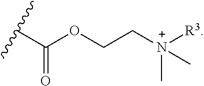

In some embodiments, Z is a bond. In some embodiments, Z is O.

In some embodiments, R¹ is —S(O)₂R^{b}. In some embodiments, R^{b} is —NR^{c}R^{d}. In some embodiments, R^{c} and R^{d} are each hydrogen.

In some embodiments, R² is halomethyl. In some embodiments, R² is fluoromethyl.

In some embodiments, R³ is halomethyl. In some embodiments, R³ is fluoromethyl.

In some embodiments, X⁻ is an anion selected from a halide and a carboxylate. In some embodiments, X⁻ is selected from chloride and bromide.

In some embodiments, the compound is selected from:

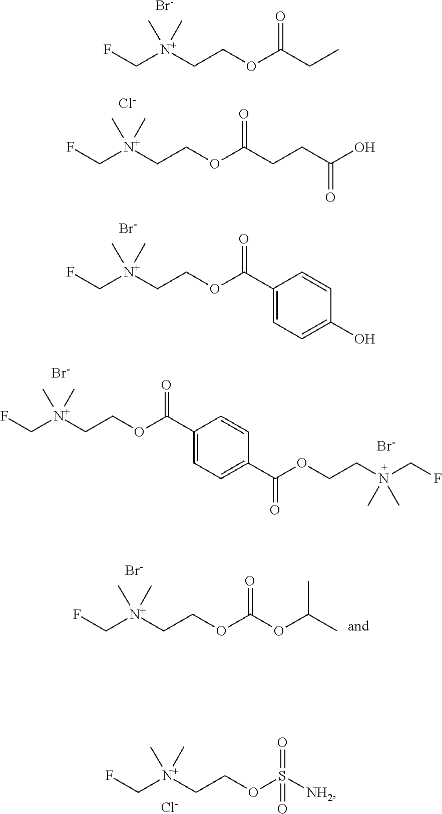

and pharmaceutically acceptable salts thereof.

In another aspect, disclosed herein is a pharmaceutical composition comprising a compound disclosed herein (e.g., a compound of formula (I)), or a pharmaceutically acceptable salt thereof, and a pharmaceutically acceptable carrier.

In another aspect, disclosed herein is a method of treating a condition associated with conversion of choline to trimethylamine in a subject in need thereof, comprising administering to the subject an effective amount of a compound disclosed herein (e.g., a compound of formula (I)), or a pharmaceutically acceptable salt thereof.

In some embodiments, the condition is selected from a cardiovascular disease, trimethylaminuria, reduced or impaired kidney function, kidney disease, diabetes mellitus, and obesity. In some embodiments, the condition is a cardiovascular disease selected from acute coronary syndrome, angina, arrhythmia, arterial aneurysm, atherosclerosis, cardiomyopathy, congestive heart failure, coronary artery disease, carotid artery disease, endocarditis, coronary thrombosis, myocardial infarction, high blood pressure/hypertension, hypercholesterolemia/hyperlipidemia, peripheral artery disease, and stroke. In some embodiments, the cardiovascular disease is due to oral biofilm formation and/or periodontal disease. In some embodiments, the condition is a kidney disease selected from chronic kidney disease and end-stage renal disease. In some embodiments, the condition is adverse ventricular remodeling, ventricular systolic dysfunction, ventricular diastolic dysfunction, cardiac dysfunction, or ventricular arrhythmia.

In another aspect, disclosed herein is a method of improving or maintaining cardiovascular health in a subject in need thereof, comprising administering to the subject an effective amount of a compound disclosed herein (e.g., a compound of formula (I)), or a pharmaceutically acceptable salt thereof.

In another aspect, disclosed herein is a method of inhibiting conversion of choline to trimethylamine in a subject in need thereof, comprising administering to the subject an effective amount of a compound disclosed herein (e.g., a compound of formula (I)), or a pharmaceutically acceptable salt thereof.

In some embodiments, the subject has an elevated level of trimethylamine N-oxide (TMAO) in blood, plasma, serum, urine, or any combination thereof.

In another aspect, disclosed herein is a method of reducing a trimethylamine N-oxide level in a subject in need thereof, comprising administering to the subject an effective amount of a compound disclosed herein (e.g., a compound of formula (I)), or a pharmaceutically acceptable salt thereof.

In some embodiments, the subject has an elevated level of trimethylamine N-oxide (TMAO) in blood, plasma, serum, urine, or any combination thereof.

In some embodiments, the methods disclosed herein further comprise administering a second therapeutic agent to the subject. In some embodiments, the second therapeutic agent is selected from Omega 3 oil, salicylic acid, dimethylbutanol, garlic oil, olive oil, krill oil, Co enzyme Q-10, a probiotic, a prebiotic, dietary fiber, psyllium husk, bismuth salts, phytosterols, grape seed oil, green tea extract, vitamin D, antioxidants, turmeric, curcumin, and resveratrol.

Other aspects and embodiments will become apparent in light of the following description.

### BRIEF DESCRIPTION OF THE DRAWINGS

FIG. 1 shows data from an in vivo d₉-choline challenge model for certain compounds disclosed herein, as described in Example 3.

FIG. 2 shows data from an in vivo Q24 hour model, as described in Example 4.

### DETAILED DESCRIPTION

The present disclosure is based, at least in part, on the discovery that compounds of formula (I) inhibit choline metabolism by gut microbiota, resulting in reduction of TMA formation. Accordingly, the disclosure provides compounds, compositions, and methods for, e.g., inhibiting conversion of choline to TMA in vitro and in vivo, for improving or maintaining cardiovascular, cerebrovascular, or peripherovascular health, and for treating disorders associated with conversion of choline to TMA.

### Definitions

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein and in the appended claims, the singular forms “a,” “an,” and “the” include plural reference unless the context clearly dictates otherwise.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “alkyl” refers to a radical of a straight or branched saturated hydrocarbon chain. The alkyl chain can include, e.g., from 1 to 24 carbon atoms (C₁-C₂₄ alkyl), 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 1 to 3 carbon atoms (C₁-C₃ alkyl), or 1 to 2 carbon atoms (C₁-C₂ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkenyl” refers to a radical of a straight or branched hydrocarbon chain containing at least one carbon-carbon double bond and no triple bonds. The double bond(s) may be located at any position(s) with the hydrocarbon chain. The alkenyl chain can include, e.g., from 2 to 24 carbon atoms (C₂-C₂₄ alkenyl), 2 to 16 carbon atoms (C₂-C₁₆ alkenyl), 2 to 14 carbon atoms (C₂-C₁₄ alkenyl), 2 to 12 carbon atoms (C₂-C₁₂ alkenyl), 2 to 10 carbon atoms (C₂-C₁₀ alkenyl), 2 to 8 carbon atoms (C₂-C₈ alkenyl), 2 to 6 carbon atoms (C₂-C₆ alkenyl), 2 to 4 carbon atoms (C₂-C₄ alkenyl), 2 to 3 carbon atoms (C₂-C₃ alkenyl), or 2 carbon atoms (C₂ alkenyl). Representative examples of alkenyl include, but are not limited to, ethenyl, 1-propenyl, 2-propenyl, 1-butenyl, 2-butenyl, butadienyl, 2-methyl-2-propenyl, 3-butenyl, pentenyl, pentadienyl, hexenyl, heptenyl, octenyl, octatrienyl, and the like.

As used herein, the term “alkynyl” means a radical of a straight or branched hydrocarbon chain containing at least one carbon-carbon triple bond. The alkynyl chain can include, e.g., from 2 to 24 carbon atoms (C₂-C₂₄ alkynyl), 2 to 16 carbon atoms (C₂-C₁₆ alkynyl), 2 to 14 carbon atoms (C₂-C₁₄ alkynyl), 2 to 12 carbon atoms (C₂-C₁₂ alkynyl), 2 to 10 carbon atoms (C₂-C₁₀alkynyl), 2 to 8 carbon atoms (C₂-C₈ alkynyl), 2 to 6 carbon atoms (C₂-C₆ alkynyl), 2 to 4 carbon atoms (C₂-C₄ alkynyl), 2 to 3 carbon atoms (C₂-C₃ alkynyl), or 2 carbon atoms (C₂ alkynyl). The triple bond(s) may be located at any position(s) with the hydrocarbon chain. Representative examples of alkynyl include, but are not limited to, ethynyl, 1-propynyl, 2-propynyl, 1-butynyl, 2-butynyl, and the like.

As used herein, the term “alkoxy” refers to an alkyl group, as defined herein, appended to the parent molecular moiety through an oxygen atom. Representative examples of alkoxy include, but are not limited to, methoxy, ethoxy, n-proxy, isopropoxy, n-butoxy, and tert-butoxy.

As used herein, the term “aryl” refers to a radical of a monocyclic, bicyclic, or tricyclic 4n+2 aromatic ring system (e.g., having 6, 10, or 14 π electrons shared in a cyclic array) having 6-14 ring carbon atoms and zero heteroatoms (“C₆-C₁₄ aryl”). In some embodiments, an aryl group has six ring carbon atoms (“C₆ aryl,” i.e., phenyl). In some embodiments, an aryl group has ten ring carbon atoms (“C₁₀ aryl,” e.g., naphthyl such as 1-naphthyl and 2-naphthyl). In some embodiments, an aryl group has fourteen ring carbon atoms (“C₁₄ aryl,” e.g., anthracenyl and phenanthrenyl).

As used herein, the term “arylalkyl” refers to an alkyl group, as defined herein, in which at least one hydrogen atom is replaced with an aryl group, as defined herein. Representative examples of arylalkyl include, but are not limited to, benzyl and phenethyl.

As used herein, the term “cycloalkyl” refers to a radical of a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “halogen” or “halo” refers to F, Cl, Br, or I.

As used herein, the term “haloalkyl” refers to an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen. Representative examples of haloalkyl include, but are not limited to, fluoromethyl, difluoromethyl, trifluoromethyl, 2-fluoroethyl, 2,2,2-trifluoroethyl, and 3,3,3-trifluoropropyl.

As used herein, the term “hydroxy” refers to an —OH group.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, thiol, thione, or combinations thereof.

As used herein, in chemical structures the indication:

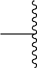

represents a point of attachment of one moiety to another moiety (e.g., a substituent group to the rest of the compound).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

When substituent groups are specified by their conventional chemical formulae, written from left to right, such indication also encompass substituent groups resulting from writing the structure from right to left. For example, if a bivalent group is shown as —CH₂O—, such indication also encompasses —OCH₂—; similarly, —OC(O)NH— also encompasses —NHC(O)O—.

The terms “administer,” “administering,” or “administration,” as used herein refer to implanting, absorbing, ingesting, injecting, inhaling, or otherwise introducing a compound or a pharmaceutical composition.

As used herein, the terms “condition,” “disease,” and “disorder” are used interchangeably.

An “effective amount” of a compound or composition refers to an amount sufficient to elicit a desired biological response (e.g., treating a condition). As will be appreciated by those skilled in the art, the effective amount of a compound may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of a compound or composition may reduce tumor burden or stop the growth or spread of a tumor.

A “therapeutically effective amount” of a compound or composition is an amount sufficient to provide a therapeutic benefit in the treatment of a condition, or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, that provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “subject” to which administration is contemplated includes, but is not limited to, a human (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys).

As used herein, the terms “treatment,” “treat,” and “treating” refer to reversing, alleviating, delaying the onset of, or inhibiting the progress of a disease or condition, or one or more signs or symptoms thereof. In some embodiments, “treatment,” “treat,” and “treating” require that signs or symptoms of the disease disorder or condition have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

### Compounds

Disclosed herein are compounds of formula (I):

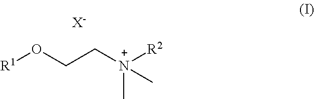

and pharmaceutically acceptable salts thereof, wherein:
R¹ is selected from —C(O)ZR^{a} and —S(O)₂R^{b};
Z is selected from a bond, —O—, and —NH—;
R^{a} is selected from alkyl, alkenyl, alkynyl, aryl, and arylalkyl, each of which is optionally substituted with 1, 2, or 3 substituents independently selected from hydroxy, —COOH, alkyl, alkoxy, halo, haloalkyl, and a group

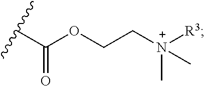

R^{b} is selected from alkyl, alkenyl, alkynyl, aryl, arylalkyl, and —NR^{c}R^{d};
R^{c} and R^{d} are each independently selected from hydrogen, alkyl, alkenyl, alkynyl, aryl, and arylalkyl;
R² and R³ are each independently selected from halomethyl and C₃-C₆ cycloalkyl; and
X⁻ is an anion.

In some embodiments, R¹ is —C(O)ZR^{a}. In some embodiments, R^{a} is selected from C₁-C₆ alkyl and aryl, each of which is unsubstituted or substituted with one substituent selected from hydroxy, —COOH, alkyl, alkoxy, halo, haloalkyl, and a group

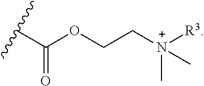

In some embodiments, R^{a} is selected from C₁-C₆ alkyl and aryl, each of which is unsubstituted or substituted with one substituent selected from hydroxy, —COOH, and a group

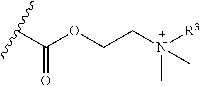

(e.g., wherein R³ is halomethyl, such as fluoromethyl).

In some embodiments, R^{a} is C₁-C₆ alkyl, which is unsubstituted or substituted with one —COOH group. In some embodiments, R^{a} is unsubstituted C₂-C₄ alkyl (e.g., ethyl or isopropyl). In some embodiments, R^{a} is C₁-C₄ alkyl substituted with one —COOH group. In some embodiments, R^{a} is C₂-C₄ alkyl substituted with one —COOH group.

In some embodiments, when R^{a} is C₁ alkyl (i.e., methyl), it is substituted with at 1, 2, or 3 substituents independently selected from hydroxy, —COOH, alkyl, alkoxy, halo, haloalkyl, and a group

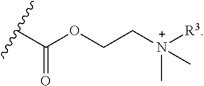

In some embodiments, R^{a} is aryl, which is unsubstituted or substituted with one substituent selected from —OH and a group

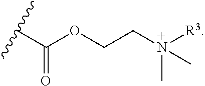

In some embodiments, R^{a} is phenyl substituted with one substituent selected from —OH and a group

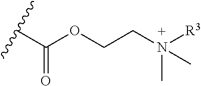

(e.g., wherein R³ is halomethyl, such as fluoromethyl).

In some embodiments, R^{a} is selected from:

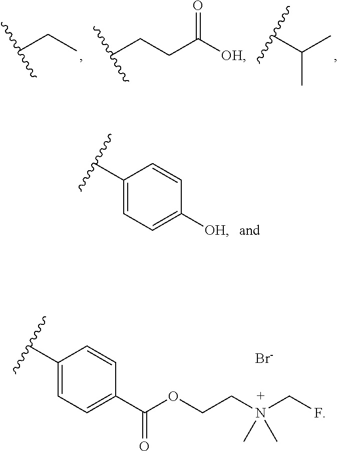

In some embodiments, Z is a bond. In some embodiments, Z is O.

In some embodiments, R¹ is —S(O)₂R^{b}. In some embodiments, R^{b} is —NR^{c}R^{d}. In some embodiments, R^{c} and R^{d} are each hydrogen.

In some embodiments, R² is halomethyl. In some embodiments, R² is fluoromethyl. In some embodiments, R² is chloromethyl. In some embodiments, R² is bromomethyl. In some embodiments, R² is iodomethyl. In some embodiments, R² is C₃-C₆ cycloalkyl. In some embodiments, R² is cyclopropyl.

In compounds of formula (I), X⁻ is an anion. While any suitable anion can be used, in some embodiments, X⁻ is selected from a halide and a carboxylate. In some embodiments, X⁻ is a halide, such as chloride or bromide. In some embodiments, X⁻ is a carboxylate, such as acetate or trifluoroacetate.

In some embodiments, the compound is selected from:

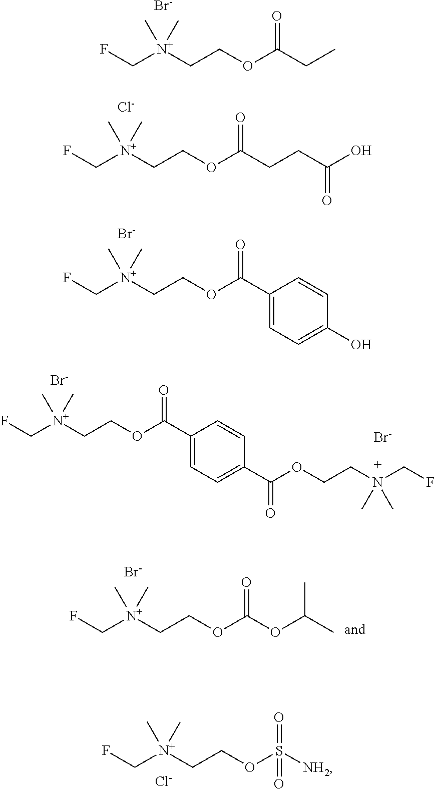

and pharmaceutically acceptable salts thereof.

The compounds of the present disclosure may have at least one asymmetric center. Additional asymmetric centers may be present depending upon the nature of the various substituent groups. Compounds with asymmetric centers give rise to enantiomers (optical isomers), diastereomers (configurational isomers) or both, and it is intended that all of the possible enantiomers and diastereomers, in mixtures and as pure or partially purified compounds, are included within the scope of this disclosure.

The independent syntheses of the enantiomerically or diastereomerically enriched compounds, or their chromatographic separations, may be achieved as known in the art by appropriate modification of the methodology disclosed herein. The absolute stereochemistry of a compound may be determined by using X-ray crystallography to determine the crystal structure of crystalline products or crystalline intermediates that are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods using chiral stationary phases, which methods are well known in the art. Alternatively, any enantiomer of a compound may be obtained by stereoselective synthesis using optically pure starting materials or reagents of known configuration by methods well known in the art.

The compounds may possess tautomeric forms, and tautomers also constitute embodiments of the disclosure.

The present disclosure also includes isotopically-labeled compounds, which are identical to those recited in formula (I) (including those compounds specifically exemplified herein), but for the fact that one or more atoms are replaced by an atom having an atomic mass or mass number different from the atomic mass or mass number usually found in nature. Examples of isotopes suitable for inclusion in the compounds of the disclosure are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ³¹P, ³⁵S, ¹⁸F, and ³⁶Cl, respectively. Substitution with heavier isotopes such as deuterium, i.e. ²H, can afford certain therapeutic advantages resulting from greater metabolic stability, for example increased in vivo half-life or reduced dosage requirements and, hence, may be particularly useful in some circumstances. The compound may incorporate positron-emitting isotopes for medical imaging and positron-emitting tomography (PET) studies for determining the distribution of receptors. Suitable positron-emitting isotopes that can be incorporated into the compounds are ¹¹C, ¹³N, ¹⁵O, and ¹⁸F. Isotopically-labeled compounds can generally be prepared by conventional techniques known to those skilled in the art, or by processes analogous to those described herein using an appropriate isotopically-labeled reagent in place of a non-isotopically-labeled reagent.

Compounds of formula (I) can be synthesized by a variety of methods. One approach is illustrated in Scheme 1, in which 2-dimethylaminoethanol is coupled to a suitable carboxylic acid or carbonic acid compound to generate an intermediate compound, that is subsequently reacted with bromofluoromethane to generate the product. Another approach is illustrated in Scheme 2, in which a suitable carboxylic acid or carbonic acid compound is directly coupled with fluoromethylcholine chloride to generate the product. Other suitable methods are disclosed in the Examples.

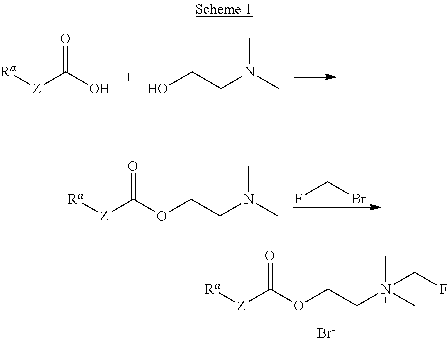

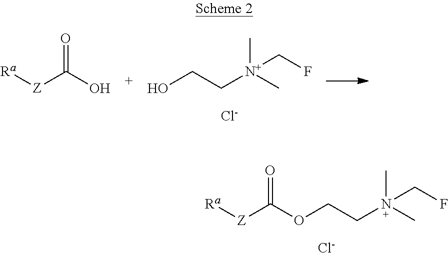

Compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Reactions can be worked up in a conventional manner, e.g., by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Standard experimentation, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006).

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the procedures described herein using a pure geometric isomer as a starting material, or by resolution of a mixture of the geometric isomers of the compound or intermediates using a standard procedure such as chromatographic separation.

The synthetic schemes and specific examples as described are illustrative and are not to be read as limiting the scope of the disclosure or the claims. Alternatives, modifications, and equivalents of the synthetic methods and specific examples are contemplated.

The disclosed compounds may exist as pharmaceutically acceptable salts. Compounds of formula (I) are already in salt form (with a positive charge on the nitrogen atom and an associated anion X⁻). Nevertheless, in some embodiments, the compound may include an additional charge and may therefore be prepared as an additional salt form. The term “pharmaceutically acceptable salt” refers to salts or zwitterions of the compounds which are water or oil-soluble or dispersible, suitable for treatment of disorders without undue toxicity, irritation, or allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water, and treated with at least one equivalent of an acid, such as hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl, and the like. In one embodiment, the compound is in the form of a halide salt, such as a chloride or bromide salt.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

Compounds disclosed herein can exist in unsolvated forms as well as solvated forms, including hydrated forms. In general, the solvated forms are equivalent to unsolvated forms and are encompassed within the scope of the present disclosure. Certain compounds of the disclosure may also exist in multiple crystalline or amorphous forms. In general, all physical forms are equivalent for the uses contemplated by the present disclosure and are intended to be within the scope of the present disclosure.

### Pharmaceutical Compositions

The disclosed compounds may be incorporated into pharmaceutical compositions suitable for administration to a subject (such as a patient, which may be a human or non-human). The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the disclosure are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease or condition, the prophylactically effective amount will be less than the therapeutically effective amount.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their pharmaceutically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90% by weight of the composition.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of Theobroma. The amount of lubricant(s) in a systemic or topical composition is typically about 5 to about 10% by weight of the composition.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50% by weight of the composition.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10% by weight of the composition.

Suitable colorants include a colorant such as an FD&C dye. When used, the amount of colorant in a systemic or topical composition is typically about 0.005 to about 0.1% by weight of the composition.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s), when used, in a systemic or topical composition is typically about 0.001 to about 1% by weight of the composition.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5% by weight of the composition.

Suitable preservatives include benzalkonium chloride, methyl paraben, and sodium benzoate. The amount of preservative(s) in a systemic or topical composition is typically about 0.01 to about 5% by weight of the composition.

Suitable glidants include silicon dioxide. The amount of glidant(s) in a systemic or topical composition is typically about 1 to about 5% by weight of the composition.

Suitable solvents include water, isotonic saline, ethyl oleate, glycerin, hydroxylated castor oils, alcohols such as ethanol, and phosphate buffer solutions. The amount of solvent(s) in a systemic or topical composition is typically from about 0 to about 100% by weight of the composition.

Suitable suspending agents include AVICEL RC-591 (from FMC Corporation of Philadelphia, PA) and sodium alginate. The amount of suspending agent(s) in a systemic or topical composition is typically about 1 to about 8% by weight of the composition.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5% by weight of the composition.

Although the amounts of components in the systemic compositions may vary depending on the type of systemic composition prepared, in general, systemic compositions include 0.01% to 50% by weight of an active compound and 50% to 99.99% by weight of one or more carriers. Compositions for parenteral administration typically include 0.1% to 10% by weight of actives and 90% to 99.9% by weight of a carrier including a diluent and a solvent.

Compositions for oral administration can have various dosage forms. For example, solid forms include tablets, capsules, granules, and bulk powders. These oral dosage forms include a safe and effective amount, usually at least about 5% by weight, and more particularly from about 25% to about 50% by weight of actives. The oral dosage compositions include about 50% to about 95% by weight of carriers, and more particularly, from about 50% to about 75% by weight.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this disclosure.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), or a pharmaceutically acceptable salt thereof), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this disclosure are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95% by weight of the composition.

Suitable propellants include propane, butane, isobutane, dimethyl ether, carbon dioxide, nitrous oxide, and combinations thereof. The amount of propellant(s) in a topical composition is typically about 0% to about 95% by weight of the composition.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95% by weight of the composition.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95% by weight of the composition.

The amount of thickener(s) in a topical composition is typically about 0% to about 95% by weight of the composition.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95% by weight of the composition.

The amount of fragrance in a topical composition is typically about 0% to about 0.5%, particularly, about 0.0010% to about 0.10% by weight of the composition.

Suitable pH adjusting additives include HCl or NaOH in amounts sufficient to adjust the pH of a topical pharmaceutical composition.

### Methods of Use

Disclosed herein are methods of reducing the production of TMA (including inhibiting the conversion of choline to TMA by a bacterium), methods of treating a condition associated with conversion of choline to TMA, methods of improving or maintaining cardiovascular health, and the like, using compounds of formula (I) (or pharmaceutically acceptable salts thereof), or compositions comprising compounds of formula (I) (or pharmaceutically acceptable salts thereof).

TMA synthesized by bacteria resident in the gut of mammals is oxidized in the liver to trimethylamine N-oxide (TMAO, TMANO). Exemplary precursors of TMA include choline, betaine, phosphatidylcholine, phosphocholine, glycerophosphocholine, carnitine (such as L-camitine), TMAO, sphingomyelin, and lecithin, many of which are derived from dietary sources such as whole eggs and beef liver. Without wishing to be bound to a particular mechanism or biochemical pathway, the anaerobic conversion of choline to TMA is facilitated by a glycyl radical enzyme homologue, choline trimethylamine-lyase (CutC). Craciun et al., Proc. Natl. Acad. Sci. (2012), 109: 21307-21312. The reduction of choline conversion to TMA by bacteria in the gut of an individual leads to a reduction in TMA absorption from the gut, leading to a subsequent reduction in plasma TMAO following oxidation of TMA to TMAO by the flavin monooxygenase 3 (FM03) enzyme in the liver. Wang et al., Nature (2011), 472: 57-63. Lower plasma TMAO levels are related to a lower incidence of major cardiovascular events in humans. Tang et al., NEJM (2013) 368: 1575-1584. The conversion of choline to TMA may be mediated by one species of bacteria or may comprise a multi-step process involving two, three or more species of bacteria.

As described previously, the present disclosure is based, at least in part, on the discovery that compounds of formula (I) interfere with choline metabolism by gut microbiota, resulting in reduction in the formation of TMA and TMAO. Accordingly, the disclosed compounds and compositions can be used in methods that, for example, inhibit the conversion of choline to TMA in vitro and in vivo, improve or maintain cardiovascular, cerebrovascular, and peripherovascular health, and treat or improve a condition associated with increased TMA and TMAO.

Disclosed herein is a method of inhibiting conversion of choline to TMA in a subject in need thereof, comprising administering to the subject an effective amount of a compound of formula (I) or a pharmaceutically acceptable salt thereof, or a composition comprising a compound of formula (I) or a pharmaceutically acceptable salt thereof. Similarly, also disclosed herein is a method of reducing a TMAO level in a subject in need thereof, comprising administering to the subject an effective amount of a compound of formula (I) or a pharmaceutically acceptable salt thereof, or a composition comprising a compound of formula (I) or a pharmaceutically acceptable salt thereof. In certain embodiments, the subject may be in need of reduced TMA levels, improvement of cardiovascular health, and the like. An individual may exhibit an elevated level of TMA or a metabolite thereof (e.g., TMAO, dimethylamine (DMA), or monomethylamine (MMA)) prior to administration, e.g., in blood, plasma, serum, urine, or the like, or any combination thereof. In various embodiments, the individual suffers from cardiovascular disease (CVD), ingests a diet high in choline, or exhibits one or more CVD risk factors (e.g., smoking, stress, high total cholesterol, high LDL cholesterol, low HDL cholesterol, age, hypertension, family history of CVD, obesity, prediabetes, diabetes, or the like).

Also disclosed herein is a method of inhibiting conversion of choline to TMA in vitro. For example, in some embodiments, the method comprises contacting a bacterium, such as a bacterium that is represented in the gut microflora, or a bacterial lysate that metabolizes choline to produce TMA, with a compound of formula (I) or a pharmaceutically acceptable salt thereof, or a composition comprising a compound of formula (I) or a pharmaceutically acceptable salt thereof. In various embodiments, the bacterium may be selected from Proteus mirabilis, Desulfovibrio alaskensis, Clostridium ljungdahlii, Clostridium scindens, Clostridium aldenense, Clostridium aminobutyricum, Collinsella tanakaei, Anaerococcus vaginalis, Streptococcus dysgalactiae, Desultitobacterium hafniense, Klebsiella variicola, Klebsiella pneumoniae, Proteus penneri, Eggerthella lenta, Edwardsiella tarda, Escherichia coli, and Escherichia fergussonii, or any combination thereof. In certain embodiments, the bacterium expresses the cutC/D gene cluster. The disclosure further provides a method of identifying a compound that inhibits TMA production. The method comprises contacting a bacterium, such as a bacterium that is part of the gut microflora, or a bacterial lysate that metabolizes choline to produce TMA with a candidate compound, such as a compound of formula (I) or a pharmaceutically acceptable salt thereof, and detecting TMA (or a metabolite thereof). In certain embodiments, the level of TMA (or metabolite thereof) produced by the bacterium in contact with the candidate compound is compared to (a) the level of TMA produced by a bacterium or lysate not contacted with a candidate compound or known TMA inhibitor or (b) the level of TMA produced by the bacterium prior to contact with the candidate compound. A reduction in the level of TMA produced by the bacterium or lysate indicates that the candidate compound inhibits conversion of choline to TMA.

It will be appreciated that “inhibiting conversion of choline to TMA” does not require complete elimination of TMA production via choline metabolism. Any reduction in TMA formation from choline or a choline-related metabolite as a precursor is contemplated, e.g., at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% reduction; and also including from about 1% to about 100%, from about 10% to about 90%, from about 20% to about 80%, from about 30% to about 70%, from about 40% to about 60%, and any combinations thereof.

In certain embodiments, the inhibition of conversion of choline to TMA by the compounds of formula (I) (or pharmaceutically acceptable salts thereof) is not brought about by an antibiotic mechanism of action, for example it is not brought about by an antibacterial mechanism of action, or by a mechanism of action which reduces cell viability to 10% or lower, when compared to vehicle control. In certain embodiments, the amount of compound needed to provide 50% inhibition of conversion of choline to TMA is less than the amount of compound that reduces cell viability to 10% or lower, when compared to vehicle control.

Any suitable method for measuring TMA in vitro or in vivo can be used in the context of the methods disclosed herein. TMA, metabolites of TMA (including TMAO, DMA, or MMA), stable isotopes of TMA (such as deuterium labeled TMA, such as d₃-, d₆-, or d₉-TMA), stable isotopes of TMAO (such as deuterium labeled TMAO, such as d₃-, d₆-, or d₉-TMAO), stable isotopes of DMA (such as deuterium labeled DMA, such as d₃-, or d₆-DMA), stable isotopes of MMA (such as deuterium labeled MMA, such as d₃-MMA), or choline (including stable isotopes of choline, for example d₉-choline) can be assessed quantitatively or qualitatively. Exemplary methods of detecting and quantifying TMA are described in, for example U.S. Pub. No. 2010/00285517, the disclosure of which is incorporated herein by reference in its entirety. For example, levels of TMA (or trimethylamine N-oxide (TMAO), DMA, or MMA) or choline are optionally measured via mass spectrometry, ultraviolet spectroscopy, or nuclear magnetic resonance spectroscopy. Mass spectrometers include an ionizing source (such as electrospray ionization), an analyzer to separate the ions formed in the ionization source according to their mass-to-charge (m/z) ratios, and a detector for the charged ions. In tandem mass spectrometry, two or more analyzers are included. Such methods are standard in the art and include, for example, HPLC with on-line electrospray ionization (ESI) and tandem mass spectrometry.

In various embodiments, TMA or TMAO is measured in a biological sample from an individual. Biological samples include, but are not limited to, whole blood, plasma, serum, urine, feces, saliva, sweat, vaginal fluid, gingival crevicular fluid, or tissue. The sample may be collected using any clinically-acceptable practice and, if desired, diluted in an appropriate buffer solution, heparinized, concentrated, or fractionated. Any of a number of aqueous buffer solutions at physiological pH, such as phosphate, Tris, or the like, can be used. Acidified buffers also may be used. For example, the final pH after adding buffer to sample may optionally be between pH 1 and pH 6, or between pH 1.5 and pH 3.0.

In addition, levels of TMA (or a metabolite or stable isotope thereof) or choline in the biological sample may be compared to a control value. The control value utilized will depend on the embodiment of the invention. In certain embodiments, the control value may be the level of TMA or TMAO produced in the individual (or by the bacterium) prior to administration or exposure to a compound of formula (I) (or a pharmaceutically acceptable salt thereof). In addition, the control value may be based on levels measured in comparable samples obtained from a reference group, such as a group of individuals from the general population, individuals diagnosed with a CVD or other TMA-associated condition, individuals not previously diagnosed with a TMA-associated condition, nonsmokers, and the like, who have not been exposed to a compound of formula (I) (or a pharmaceutically acceptable salt thereof). Levels of TMA or TMAO or choline may be compared to a single control value or to a range of control values. An individual is optionally identified as having an enhanced level of TMA prior to administration by comparing the amount of TMA in a biological sample from the individual with a control value.

Also disclosed herein are methods of improving cardiovascular health of in a subject in need thereof, comprising administering to the subject an effective amount of a compound of formula (I) (or a pharmaceutically acceptable salt thereof), or a composition comprising a compound of formula (I) (or a pharmaceutically acceptable salt thereof). In some embodiments, cardiovascular health is assessed by testing arterial elasticity, blood pressure, ankle/brachial index, electrocardiogram, ventricular ultrasound, platelet function (for example platelet aggregation), and/or blood/urine tests to measure, e.g., cholesterol, albumin excretion, C-reactive protein, and/or plasma B-type peptide (BNP) concentration. In various embodiments, administration of the compound of formula (I) (or pharmaceutically acceptable salt thereof), improves or maintains one or more of the assay outcomes within normal ranges. Normal ranges of outcomes of each test are known in the art. Improvement in cardiovascular health is, in some embodiments, marked by a reduction in circulating total cholesterol levels, reduction in circulating low density lipoproteins (LDLs), reduction in circulating triglycerides, or reduction in blood pressure. In some embodiments, the subject has an elevated level of TMAO, e.g., in blood, plasma, serum, urine, or any combination thereof.

Also disclosed herein are methods of treating a condition associated with conversion of choline to TMA in a subject in need thereof, comprising administering to the subject an effective amount of a compound of formula (I) or a pharmaceutically acceptable salt thereof, or a composition comprising a compound of formula (I) or a pharmaceutically acceptable salt thereof.

In some embodiments, the condition associated with the conversion of choline to TMA is a cardiovascular disease, trimethylaminuria, reduced or impaired kidney function, kidney disease (e.g., chronic kidney disease or end-stage renal disease), trimethylaminuria, obesity, or diabetes mellitus.

In some embodiments, the condition is cardiovascular disease. The term “cardiovascular disease” (CVD) is used in the art in reference to conditions affecting the heart, heart valves, and vasculature (such as arteries and veins) of the body and encompasses diseases and conditions including, but not limited to, angina, arrhythmia, arteriosclerosis, atherosclerosis, myocardial infarction, acute coronary syndrome, cardiomyopathy, congestive heart failure, coronary thrombosis, arterial aneurysm (e.g., aortic aneurysm, iliac aneurysm, or femoral aneurysm), aortic dissection, pulmonary embolism, high blood pressure/hypertension (e.g., primary hypertension), hypercholesterolemia/hyperlipidemia, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), carotid artery disease, peripheral artery disease (PAD), cerebrovascular disease, adverse ventricular remodeling, ventricular systolic dysfunction, ventricular diastolic dysfunction, cardiac dysfunction, ventricular arrhythmia, stroke, and the like. In some embodiments, the cardiovascular disease is due to oral biofilm formation and/or periodontal disease.

In some embodiments, the condition is atherosclerosis. Atherosclerosis involves the formation of atheromatous plaques that lead to narrowing (“stenosis”) of the vasculature, which can ultimately lead to partial or complete occlusion or rupture (aneurism) of the vessel, heart failure, aortic dissection, and ischemic events such as myocardial infarction and stroke. In various non-limiting embodiments, an inventive method inhibits, reduces, or reverses (in whole or in part) the onset or progression of atherosclerosis (for example reducing or preventing hardening or thickening of the arteries, plaque formation, endothelium damage, or arterial inflammation).

In some embodiments, the condition is trimethylaminurina. Trimethylaminuria (TMAU) is a condition characterized by an inability of individuals to convert TMA to TMAO, wherein affected individuals may have a fish-like body odor present in their urine, sweat or breath. (Yamazaki et al. Life Sciences (2004) 74: 2739-2747). Such individuals may benefit from a reduction in metabolism of substrates including but not limited to choline, to TMA by bacteria in the gut. Individuals with TMAU or those wishing to reduce their levels of TMA and TMAO, may also consume activated charcoal or copper chlorophyllin, which act as sequestering agents, for example to make TMA unavailable to transfer into the blood stream of an individual. Such sequestering agents may adsorb TMA, which is then excreted from the digestive tract along with the sequestering agent.

Other conditions associated with increased levels of TMA may include production of TMA by bacteria in the vagina leading to vaginal odor, or production of TMA by bacteria on the body leading to body odor, or production of TMA by bacteria in the mouth leading to bad breath or oral care biofilm development. Furthermore, during pregnancy where the third trimester and post-partum period are associated with an increased risk of thrombosis, lowering TMA and TMAO levels may reduce this risk.

Also disclosed herein are methods of improving a condition associated with conversion of choline to TMA in a subject in need thereof, comprising administering to the subject an effective amount of a compound of formula (I) or a pharmaceutically acceptable salt thereof, or a composition comprising a compound of formula (I) or a pharmaceutically acceptable salt thereof. As used herein, “improving a condition” refers to any reduction in the severity or onset of symptoms associated with a disorder caused, at least in part, by TMA. One of ordinary skill in the art will appreciate that any degree of protection from, or amelioration of, a TMA-related disorder or symptom associated therewith is beneficial to an individual, such as a human. The quality of life of an individual is improved by reducing to any degree the severity of symptoms in an individual or delaying the appearance of symptoms. Accordingly, a method in one aspect is performed as soon as possible after it has been determined that an individual is at risk for developing a TMA-related disorder or as soon as possible after a TMA-related disorder is detected.

In various embodiments, administration of the compound formula (I) (or a pharmaceutically acceptable salt thereof) results in reduced TMA or TMAO levels, reduced total cholesterol levels, reduced LDL levels, increased HDL levels, reduced triglyceride levels, or normalized levels of other biomarkers associated with CVD (for example excreted albumin, C-reactive protein, or plasma B-type peptide (BNP)). In some embodiments, the compound of formula (I) (or a pharmaceutically acceptable salt thereof) reduces the risk of cardiovascular disease, trimethylaminuria, reduced or impaired kidney function, kidney disease (e.g., chronic kidney disease or end-stage renal disease), trimethylaminuria, obesity, or diabetes mellitus, when administered to a subject.

In some embodiments, the amount of compound or composition administered to the individual is sufficient to inhibit (in whole or in part) formation of TMA from choline. In various aspects of the disclosure, the amount improves cardiovascular health or achieves a beneficial biological response with respect to an unwanted condition associated with TMA (for instance the amount is sufficient to ameliorate, slow the progression, or prevent a condition (such as CVD)). The effect can be detected by, for example, an improvement in clinical condition, reduction in symptoms, or by any of the assays or clinical diagnostic tests described herein. The precise effective amount for an individual can depend upon the individual's body weight, size, and health; the nature and extent of the condition; and the compound or combination of agents selected for administration. In various aspects, the amount of compound administered to an individual is about 0.001 mg/kg to about 1000 mg/kg. Specific ranges of doses in mg/kg include about 0.1 mg/kg to about 500 mg/kg, about 0.5 mg/kg to about 200 mg/kg, about 1 mg/kg to about 100 mg/kg, about 2 mg/kg to about 50 mg/kg, and about 5 mg/kg to about 30 mg/kg. An effective amount may be administered to an individual as a single deployment of compound or as a divided dose (such as a single dose administered in multiple subunits contemporaneously or close in time). An amount of compound may be delivered one, two, or three times a day; one, two, or three times a week; or one, two, three, or four times a month. The compound may be delivered as a prodrug, which is converted to an active drug in vitro or in vivo.

A compound or composition can be administered by any route that allows inhibition of choline conversion to TMA. The compound or composition is, in various aspects of the invention, delivered to an individual parenterally (for example intravenously, intraperitoneally, intrapulmonary, subcutaneously or intramuscularly), intrathecally, topically, transdermally, rectally, orally, sublingually, nasally or by inhalation. In various embodiments, a compound or a composition is administered to the gastrointestinal tract, such as by ingestion. Sustained release formulations may also be employed to achieve a controlled release of the compound when in contact with body fluids in the gastrointestinal tract. Sustained release formulations are known in the art, and typically include a polymer matrix of a biological degradable polymer, a water-soluble polymer, or a mixture of both, optionally with suitable surfactants.

In some embodiments, the compounds described herein may be provided in a delayed release composition and are optionally released in a specific region of the digestive tract of an individual. For example, the composition may be provided such that the compounds are released from an orally dosed composition in the distal portion of the digestive tract such as the ileum or the colon. In certain embodiments, the delayed release composition releases the compounds at a specific pH, or at a range of pH for targeted delivery within the digestive tract of an individual. The compounds may be released, for example, between pH 6.0 and pH 9.0, between pH 6.5 and pH 8.0, between pH 6.5 and pH 7.5, between pH 7.0 and pH 7.5, or between pH 7.0 and pH 8.0.

Methods described herein may comprise administering a second agent to an individual. The term “second agent,” as used herein, serves to distinguish the agent from the compound of formula (I) (or pharmaceutically acceptable salt thereof) and is not meant to limit the number of additional agents used in a method or denote an order of administration. One or more second agents are optionally incorporated in a composition with the compound of formula (I) (or pharmaceutically acceptable salt thereof), administered concurrently but in separate dosage forms, or administered separately in time.

Exemplary second agents include, but are not limited to, antimicrobials (such as antibiotics that kill bacteria in the gut); agents that improve intestinal motility (such as fiber or psyllium); agents that further reduce TMA levels in the gut including sequestering agents (such as activated charcoal, or copper chlorophyllin); agents that further reduce TMA levels or production of TMA metabolites; agents that improve one or more aspects of cardiovascular health, such as agents that normalize blood pressure, decrease vascular inflammation, reduce platelet activation, normalize lipid abnormalities; agents that promote the excretion of TMA from the body; or agents that bind TMA so that it cannot be converted to TMAO. In various embodiments, the second agent is selected from the group consisting of an omega-3 oil, salicylic acid (aspirin), dimethylbutanol, garlic oil, garlic extract, olive oil, krill oil, coenzyme Q10, a probiotic, a prebiotic, a dietary fiber, psyllium husk, bismuth salts, phytosterols, grape seed oil, green tea extract, vitamin D, an antioxidant (such as vitamin C and vitamin E), turmeric, curcumin, resveratrol, activated charcoal, or copper chlorophyllin. In certain embodiments, the second agent is dimethylbutanol or inhibitors of the formation of TMA from precursors other than choline (for example betaine, phosphatidylcholine, crotonobetaine, or carnitine). Additional exemplary second agents are described in US 2017/0151208, US 2017/0151250, US 2017/0152222, or US 2018/0000754, which are incorporated here by reference.

Methods disclosed herein may further comprise administration of one or more cardiovascular disease therapies. Examples of therapies include, but are not limited to, statins (e.g., Lipitor™ (atorvastatin), Pravachol™ (pravastatin), Zocor™ (simvastatin), Mevacor™ (lovastatin), and Lescol™ (fluvastatin)) or other agents that interfere with the activity of HMGCoA reductase, nicotinic acid (niacin, which lowers LDL cholesterol levels), fibrates (which lower blood triglyceride levels and include, for example Bezafibrate (such as Bezalip®), Ciprofibrate (such as Modalim®), Clofibrate, Gemfibrozil (such as Lopid®) and Fenofibrate (such as TriCor®)), bile acid resins (such as Cholestyramine, Colestipol (Colestid), and Cholsevelam (Welchol)), cholesterol absorption inhibitors (such as Ezetimibe (Zetia®, Ezetrol®, Ezemibe®)), phytosterols such as sitosterol (Take Control (Lipton)), sitostanol (Benechol), or stigmastanol), alginates and pectins, lecithin, and nutraceuticals (such as extract of green tea and other extracts that include polyphenols, particularly epigallocatechin gallate (EGCG), Cholest-Arrest™ (500 mg garlic and 200 mg lecithin). Cholestaway™ (700 mg Calcium carbonate, 170 mg magnesium oxidem 50 μg chromium picolinate), Cholest-Off™ (900 mg of plant sterols/stanols), Guggul Bolic (750 mg gugulipid (Commiphora mukul gum resin), and Kyolic® (600 mg aged garlic extract and 380 mg lecithin)).

### Kits

For use in the methods described herein, kits and articles of manufacture are also provided, which include a compound or pharmaceutical composition described herein (e.g., a compound of formula (I) or a pharmaceutically acceptable salt thereof, or a pharmaceutical composition comprising a compound of formula (I) or a pharmaceutically acceptable salt thereof). In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

The articles of manufacture provided herein contain packaging materials. Packaging materials for use in packaging pharmaceutical products include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, in some embodiments the container(s) includes a compound of formula (I), or a pharmaceutically acceptable salt thereof, optionally in a composition or in combination with another agent as disclosed herein. The container(s) optionally have a sterile access port (for example the container is an intravenous solution bag or a vial having a stopper pierceable by a hypodermic injection needle). Such kits optionally comprises a compound with an identifying description or label or instructions relating to its use in one or more methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical composition is presented in a pack or dispenser device which contains one or more unit dosage forms containing a compound provided herein. The pack, for example, contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### EXAMPLES

Abbreviations used in the Examples include the following: DMSO is dimethylsulfoxide; EtOAc is ethyl acetate; HRMS is high resolution mass spectrometry; LCMS is liquid chromatography mass spectrometry; NMR is nuclear magnetic resonance; and THF is tetrahydrofuran.

### Example 1: Compound Syntheses

### Compound 1: N-(Fluoromethyl)-N,N-dimethyl-2-(propionyloxy)ethanaminium bromide

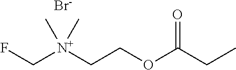

Step 1: 2-(Dimethylamino)ethyl propionate. To a solution of propionic acid (300 mg, 4.03 mmol, 1.2 equiv) in THF (10 mL) was added 1,1′-carbonyldiimidazole (654 mg, 4.03 mmol, 1.2 equiv) at room temperature. The resulting mixture was stirred at 50° C. for 2 h. 2-dimethylaminoethanol (300 mg, 3.36 mmol, 1.0 equiv) was then added and the resulting mixture stirred at 50° C. for 16 h. After the reaction was complete, the solvent was removed under reduced pressure. The residue was purified by flash chromatography eluting with 0-10 percent methanol-dichloromethane to afford the product (130 mg, 34.8%) as colorless oil. LCMS (API-ES): m/z 146.2 [M+H]⁺

Step 2. N-(Fluoromethyl)-N,N-dimethyl-2-(propionyloxy)ethanaminium bromide To a solution of 2-(dimethylamino)ethyl propionate (130 mg, 0.90 mmol) in acetonitrile (3 mL) was added bromofluoromethane (121 mg, 1.08 mmol, 1.2 equiv). The resulting mixture was stirred at room temperature for 48 h and then concentrated under reduced pressure to dryness. The resulting residue was lyophilized to give the product (175 mg, 75.8%) as colorless oil. ¹H-NMR (400 MHz, DMSO-d₆): δ 5.51 (d, J_{H,F}=44.8 Hz, 2H), 4.40-4.52 (m, 2H), 3.72-3.82 (m, 2H), 3.12-3.24 (m, 6H), 2.37 (q, J=7.6 Hz, 2H), 1.05 (t, J=7.6 Hz, 3H) LCMS (API-ES): C₈H₁₇FNO₂⁺ m/z=178.0. High resolution mass spec: Calculated for C₈H₁₇FNO₂⁺, 178.1238; found, 178.1233

### Compound 2: 2-((3-carboxypropanoyl)oxy)-N-(fluoromethyl)-N,N-dimethylethan-1-aminium chloride

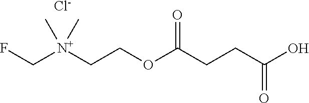

Fluoromethylcholine (1.1460 g, 7.2711 mmol) and succinic anhydride (4.1773 g, 41.744 mmol) were combined in a 50 mL round bottom flask with a stir bar and the resulting mixture heated using a heat gun until the succinic anhydride melted (˜120° C.). Once the succinic anhydride melted, fluoromethylcholine started incorporating into the melt. Stirring was initiated and heat was maintained using a heat gun at a distance of 4-6 inches. The flask was heated for 20 s and then heat removed for 15 s and this heating process alternated for ˜10 min. The reaction mixture turned dark brown. The reaction mixture was cooled to room temperature to give a brown solid. Brown solid was broken up using a spatula and then washed with acetone (5×25 mL). Washings discarded and resulting brown solid dried under high vacuum to give 2-((3-carboxypropanoyl)oxy)-N-(fluoromethyl)-N,N-dimethylethan-1-aminium chloride (FMC succinate). 1.7475 g yield (93%). ¹H-NMR (400 MHz, D₂O): δ 5.36 (d, J_{H,F}=44.9 Hz, 2H), 4.50-4.58 (m, 2H), 3.75-3.83 (m, 2H), 3.19 (apparent d, 6H), 2.61-2.72 (m, 4H). ¹³C-NMR (100 MHz, D₂O): δ 176.9, 173.8, 96.6 (dt, J_{C,F}=221 Hz), 60.1, 57.9, 47.9 (2C), 28.7, 28.6. HRMS (ESI) m/z: [M−Cl]⁺ calculated for C₉H₁₇FNO₄⁺, 222.1136; found, 222.1126.

### Compound 3: N-(fluoromethyl)-2-((4-hydroxybenzoyl)oxy)-N,N-dimethylethan-1-aminium bromide

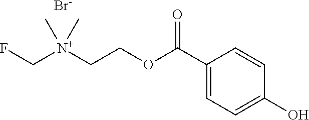 [SMILES: [Br-] ; C[N+](C)(CF)CCOC(=O)c1ccc(O)cc1]

Step 1: 2-(Dimethylamino)ethyl 4-(benzyloxy)benzoate. To a solution of 4-(benzyloxy)benzoic acid (300 mg, 1.31 mmol) in tetrahydrofuran (4 mL) was added 1,1′-carbonyldiimidazole (320 mg, 1.97 mmol, 1.5 equiv). The reaction mixture was stirred at room temperature for 1 h under nitrogen and then 2-dimethylaminoethanol (117 mg, 1.31 mmol, 1.0 equiv) was added in one portion. The reaction mixture was stirred at room temperature for 18 h under nitrogen and then concentrated. The residue was purified using silica gel column chromatography eluting with 0-80% EtOAc-Petroleum ether to give 2-(dimethylamino)ethyl 4-(benzyloxy)benzoate (266 mg, 67.8%) as a colorless oil. LCMS (API-ES): >95% (214, 254 nm), m/z=300.0 [M+H]⁺

Step 2: 2-(Dimethylamino)ethyl 4-hydroxybenzoate. To a solution of 2-(dimethylamino)ethyl 4-(benzyloxy)benzoate (260 mg, 0.87 mmol) in methanol (8 mL) was added Pd/C (52 mg, 20%). The reaction mixture was stirred at room temperature for 4 h under hydrogen. Upon completion, the reaction mixture was filtered and the filtrate concentrated. The residue was purified by prep-TLC to give 2-(dimethylamino)ethyl 4-hydroxybenzoate (96 mg, 52.8%) as a white solid. LCMS (API-ES): >95% (214, 254 nm), m/z=210.0 [M+H]⁺

Step 3: N-(fluoromethyl)-2-((4-hydroxybenzoyl)oxy)-N,N-dimethylethan-1-aminium bromide. To a solution of 2-(dimethylamino)ethyl 4-hydroxybenzoate (96 mg, 0.46 mmol) in acetonitrile (1.5 mL) was added bromofluoromethane (62 mg, 0.55 mmol, 1.2 equiv) at 0° C. under nitrogen. The reaction mixture was stirred at room temperature for 48 h. Upon completion, the reaction mixture was concentrated and diluted with ethyl acetate (5 mL). The mixture was filtered and dried to give the product N-(fluoromethyl)-2-((4-hydroxybenzoyl)oxy)-N,N-dimethylethan-1-aminium bromide (97.9 mg, 66.2%) as a white solid. ¹H-NMR (400 MHz, D₂O): δ 7.88 (d, J=8.4 Hz, 2H), 6.89 (d, J=8.4 Hz, 2H), 5.42 (d, J_{H,F}=44.8 Hz, 2H), 4.70-4.80 (m, 2H), 3.86-3.96 (m, 2H), 3.24 (s, 6H). LCMS (API-ES): >99% (214, 254 nm), C₁₂H₁₇FNO₃⁺ m/z=242.0. High resolution mass spec: Calculated for C₁₂H₁₇FNO₃⁺, 242.1187; found, 242.1181.

### Compound 4: 2,2′-(Terephthaloylbis(oxy))bis(N-(fluoromethyl)-N,N-dimethylethan-1-aminium) bromide

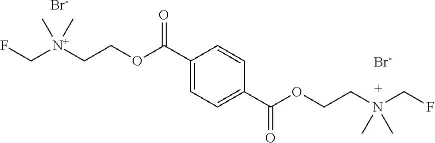

Step 1: Bis(2-(dimethylamino)ethyl) terephthalate. To a solution of terephthalic acid (300 mg, 1.81 mmol) in tetrahydrofuran (10 mL) was added 1,1′-carbonyldiimidazole (732 mg, 4.51 mmol, 2.5 equiv). The reaction mixture was stirred at room temperature for 1 h under nitrogen and then 2-dimethylaminoethanol (354 mg, 3.97 mmol, 2.2 equiv) was added. The reaction mixture was stirred at room temperature for 18 h under nitrogen and then concentrated. The residue was purified using silica gel column chromatography eluting with 0-60% ethyl acetate-petroleum ether to give bis(2-(dimethylamino)ethyl) terephthalate (77 mg, 6.3%) as a white solid. LCMS (API-ES): >95% (214, 254 nm), m/z=309.0 [M+H]⁺.

Step 2: 2,2′-(Terephthaloylbis(oxy))bis(N-(fluoromethyl)-N,N-dimethylethan-1-aminium) bromide. To a solution of bis(2-(dimethylamino)ethyl) terephthalate (77 mg, 0.25 mmol) in acetonitrile (1.5 mL) was added bromofluoromethane (77 mg, 0.63 mmol, 2.5 equiv) at 0° C. under nitrogen. The reaction mixture was stirred at room temperature for 48 h and then concentrated and diluted with ethyl acetate (5 mL). The mixture was filtered and dried to afford the compound 2,2′-(terephthaloylbis(oxy))bis(N-(fluoromethyl)-N,N-dimethylethan-1-aminium) bromide (78.9 mg, 57.0%) as a white solid. ¹H-NMR (400 MHz, D₂O) δ 8.08 (s, 4H), 5.50 (s, 2H), 5.39 (s, 2H), 4.77-4.84 (m, 4H), 3.92-4.00 (m, 4H), 3.27 (s, 12H). LCMS (API-ES): >99% (214, 254 nm), C₁₈H₂₈F₂N₂O₄⁺² m/z=187.0.

### Compound 5: N-(Fluoromethyl)-2-((isopropoxycarbonyl)oxy)-N,N-dimethylethanaminium bromide

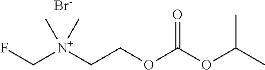

Step 1: 2-(Dimethylamino)ethyl isopropyl carbonate. To a solution of 2-(dimethylamino)ethanol (300 mg, 3.36 mmol) in THF (10 mL) was added sodium hydride (162 mg, 4.04 mmol, 1.2 equiv) at 0° C. The mixture was stirred at room temperature for 2 h and then chloromethyl isopropyl carbonate (796 mg, 4.04 mmol, 1.2 equiv) was added and resulting mixture stirred at room temperature for 2 h. The reaction was quenched with water (50 mL) and extracted with dichloromethane (2×50 mL). The combined organics were washed with brine (50 mL), dried over anhydrous sodium sulfate, filtered and concentrated. The residue was purified by flash chromatography eluting with 0-10% methanol-dichloromethane to give the product (200 mg, 28.9%) as colorless oil. LCMS (API-ES): m/z 176.2 [M+H]⁺

Step 2: N-(Fluoromethyl)-2-((isopropoxycarbonyl)oxy)-N,N-dimethylethanaminium bromide. To a solution of 2-(dimethylamino)ethyl isopropyl carbonate (200 mg, 1.14 mmol) in acetonitrile (3 mL) was added bromofluoromethane (155 mg, 1.37 mmol, 1.2 equiv). The resulting mixture was stirred for 48 h at room temperature. The reaction mixture was concentrated under reduced pressure to dryness to give the product (240 mg, 72.9%) as colorless oil. ¹H NMR (400 MHz, DMSO-d₆) δ 5.51 (d, J=45.2 Hz, 2H), 4.80 (septet, J=6.2 Hz, 1H), 4.44-4.60 (m, 2H), 3.80 (t, J=4.0 Hz, 2H), 3.18 (s, 3H), 3.17 (s, 3H), 1.25 (d, J=6.2 Hz, 6H). LCMS (API-ES): C₉H₁₉FNO₃⁺ m/z=208.0. HRMS (ESI) m/z: [M−Br]⁺ calculated for C₉H₁₉FNO₃⁺ 208.1343, found 208.1341

### Compound 6: N-(fluoromethyl)-N,N-dimethyl-2-(sulfamoyloxy)ethan-1-aminium chloride

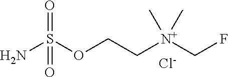

Sulfamoyl chloride (1.4797 g, 12.38 mmol) was added to a stirred solution of fluoromethylcholine chloride (0.7802 g, 4.95 mmol) in NMP (8 mL) at 0° C. (ice bath). Resulting mixture stirred at 0° C. for 15 min then allowed to warm to room temp while stirring for 80 min. Reaction mixture was poured into stirred 100 mL EtOAc whereupon a white suspension formed. Stirring was stopped, and the suspension was allowed to settle overnight. The clear solution was decanted off leaving behind a gummy solid. The gummy solid was treated with iPrOH (˜30 mL) and let sit for 2 days. iPrOH decanted off to leave behind a white solid. White solid re-crystallized using a minimum amount of hot EtOH (˜150 mL) to give short white needles upon cooling to room temperature (0.7663 g); 65% yield. ¹H-NMR (400 MHz, D₂O): δ 5.39 (d, J_{H,F}=44.7 Hz, 2H), 4.60-4.67 (m, 2H), 3.86-3.93 (m, 2H), 3.23 (apparent d, 6H). ¹³C-NMR (100 MHz, D₂O): δ 96.7 (dt, J_{C,F}=222 Hz), 62.9, 60.1, 48.1 (2C). HRMS (ESI) m/z: [M−Cl]⁺ calculated for C₅H₁₄FN₂O₃S⁺ 201.0704, found 201.0694.

### Example 2: In Vitro TMA Inhibition

Full-length, recombinant CutC/D was expressed through the use of competent E. coli cells transformed with the expression plasmids encoding for the cutC and cutD genes from P. mirabilis as described (Wang Z et al., Cell 2015 Dec. 17; 163(7):1585-95). CutC/D over-expressing cells were grown in nutrient-rich LB medium with the addition of ampicillin (100 μg/mL). The cell pellets were kept on ice and re-suspended in the lysis buffer: 200 mM HEPES, 50 mM NaCl, 10 mM MgCl₂, pH 8.0. Clarified lysate was aliquoted and flash frozen for storage at −80° C. until use.

Choline TMA lyase enzymatic activity assays were conducted to measure the conversion of isotope-labeled d₉-choline to d₉-TMA. Upon thawing an aliquot of frozen clarified lysate on ice and gentle mixing, clarified lysate was diluted to 5 mg/mL in lysis buffer and the necessary co-factors were added (final concentration: 1 mM SAM, 10 mM NaDT, and 2 mM NADH). Potential inhibitor compounds (typically dissolved in H₂O) were serially diluted (10-fold) and added to individual reaction mixtures (done in triplicate). The mixture was then allowed to incubate for at least 15 minutes at 22° C. before the addition of substrate (300 μM final d₉-choline) to initiate the reaction, which was carried out for 2 hours at room temperature (22° C.) in the dark in gas tight sealed reaction vials. The reactions were quenched with the addition of 200 μL of 1 M NaOH and 0.5 μM of the internal standard ([¹³C₃¹⁵N₁]TMA), vortexing and placement in ice. d₉-TMA production was quantified using mass spectrometry by measuring the peak area ratio of the precursor to product ion transition of d₉-TMA (m/z 69→49) to the internal standard [¹³C₃¹⁵N₁]TMA (m/z 64→47).

Calculated IC₅₀ values are provided in Table 1.

TABLE 1
IC₅₀ Values

| Cpd. No. | IC₅₀ (nM) |
|---|---|
| 1 | 4200 |
| 2 | 2400 |
| 3 | 3600 |
| 4 | 86 |
| 5 | 4200 |

### Example 3: In Vivo d₉-Choline Challenge Model—q3 hr

In this model, the indicated drug and isotope tracer labeled choline (d₉-choline) were provided via oral gavage concomitantly (like giving inhibitor at same time of a glucose tolerance test), while animals were maintained ad lib on a 10% Choline diet (average plasma TMAO levels on 1% Choline added diet are 201.7+/−104.5 μM, n=121), and plasma was recovered at the indicated times post gavage to monitor the impact of inhibitor on d₉-TMA and d₉-TMAO generation. This model helps to define whether an agent can inhibit TMA and TMAO production when given orally, provided it has PK properties that permit it to reach the gut microbiota compartment within the time frame monitored.

Briefly, C57bl/6 Female mice (Jackson #0664) ≥8 wks of age were fasted for 1 hr in a clean empty cage with water ad libitum. Mice were given a single bolus of 2 mg d₉-choline and the indicated dose (in mg/kg) of Compound in 0.2 mL of sterile water. Blood was collected at the indicated time point(s). Plasma was analyzed for d₉-TMA and d₉-TMAO production by stable isotope dilution tandem mass spectrometry. After observing near comparable results (in terms of calculated ED50) when looking at multiple different time points post gavage (1.5, 3 and 4.5 hr), results are reported at peak levels (3 h post gavage). Results were plotted using GraphPad Prism (v9.1) in comparison to either a same-day control (n=4-5) or a compiled control (n=15-24 mice, across 5 independent experiments) and a Mann Whitney test was conducted.

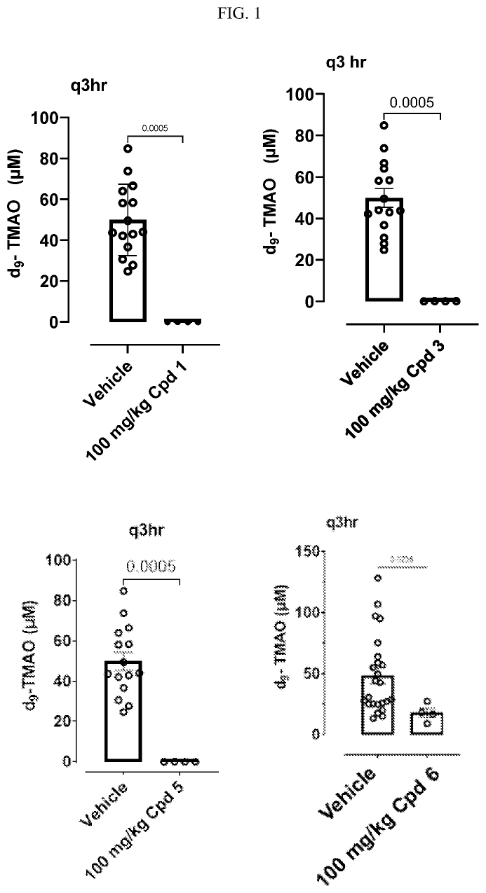

The plotted data is shown in FIG. 1.

### Example 4: In Vivo Q24 hr Model

In this model, drug was provided via oral gavage once daily in an animals placed on a high choline diet ad libitum, and plasma was recovered on a daily (Q24 h) basis to monitor therapeutic response (TMAO level) at time of trough blood level (just before gavage). This model can help to define minimal daily oral regimen to achieve desired TMAO reduction throughout the entire duration of study.

Briefly, C57bl/6 Female mice (Jackson #0664, ≥8 wks of age) were maintained prior to initiation of the study on a 10% Choline diet for at least 48 hrs. At the start of this study, mice were given the indicated dose (in mg/kg) of compound via oral gavage (typically in 0.2 mL of sterile water or vehicle). Blood was collected prior to gavage (baseline) and q24 hr after gavage for analysis of TMA, TMAO and creatinine. Data was plotted in Graphpad prism (v9) and a paired t-test (Wilcoxon) was completed, p<0.05 was considered significant.

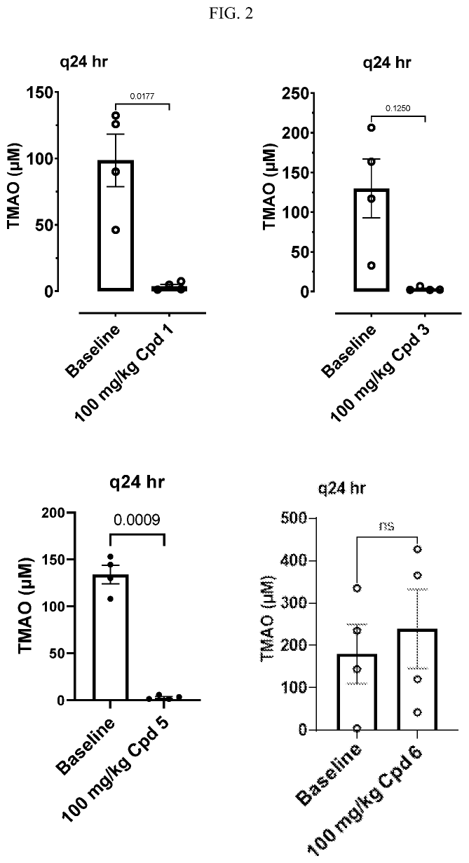

The plotted data is shown in FIG. 2.

### Example 5: Pharmacokinetic Data

Female mice (Jackson #00664 or Taconic Swiss Webster) at least 8 weeks of age were placed on 1% choline added diet (Teklad, TD.09041) ad libitum. After at least 48 hours on diet, animals were given a bolus gavage of 0.2 mL aqueous solution (2 mg d₉-choline+100 mg/kg (TEST COMPOUND)). After gavage, animals were singly-housed in a cage fitted with collection wires on the cage floor. Blood and urine samples were collected prior to gavage, 1 hour, 2 hours, 3 hours, 4 hours, and 24 hours after gavage. Blood was collected via saphenous vein. Urine was collected by manual expression of the bladder. Feces were pooled from the cage floor at the end of the 24 hours.

Analysis of plasma: 15 μL mouse plasma was aliquoted into a 1.5 mL Eppendorf tube and 60 μL cold methanol containing 10 μM d₉-choline and d₉-betaine as internal standards was added and vortexed for one minute. The samples were spun down at 20000 g, at 4° C. for 10 minutes and then 60 μL supernatant was transferred to a 2 mL mass spec vial with a plastic insert for LC/MS/MS analysis.

Analysis of urine: Urine was diluted 20-fold in water and then 15 μL of this dilution was aliquoted to a 1.5 mL Eppendorf tube. 60 μL cold methanol containing 10 μM d₉-choline and d₉-betaine as internal standards was added to the tube and the resulting mixture vortexed for one minute. The samples were spun down at 20000 g, 4° C. for 10 minutes and then 60 μL of the supernatant was transferred to a 2 mL mass spec vial with plastic insert for LC/MS/MS analysis. Serial concentrations of standards were mixed with 4 volumes of 10 μM d₉-choline and d₉-betaine to prepare a standard curve.

Analysis of feces: Feces were homogenized in 10 volumes water (assuming 1 mg feces is 1 μL in volume) containing 10 μM d₉-choline and d₉-betaine as internal standards followed by centrifugation at 6000 g, 4° C. for 12 minutes. 200 μL supernatant was filtered through a 3K cut-off filter (Amicon® Ultra-0.5 mL centrifugal filters). The filtrate was collected in a 2 mL mass spec vial with a plastic insert. Serial concentrations of standards were mixed with 10 volumes of 10 μM d₉-choline and d₉-betaine to prepare a standard curve.

The prepared sample (1 μL) was analyzed by injection onto a silica column (2.0×150 mm, 5 μm Luna silica; Cat. No. 00F-4274-B0, Phenomenex, Torrance, CA) at a flow rate of 0.25 mL min-1 using a 2 Vanquish pump system (Thermo Scientific, Waltham, MA), interfaced with a TSQ mass spectrometer (Thermo Scientific, Waltham, MA). A discontinuous gradient was generated to resolve the analytes by mixing solvent A (0.2% formic acid in water) with solvent B (0.2% formic acid in methanol) at different ratios starting from 0% B linearly to 15% B over 5 min, then linearly to 100% B over 2.5 min, then hold for 3 min, and then back to 0% B. Fluoromethylcholine, fluoromethylbetaine and their corresponding internal standards, d₉-choline and d₉-betaine, were monitored using electrospray ionization in positive-ion mode with multiple reaction monitoring (MRM) of precursor and characteristic product-ion transitions of m/z 122→58; 136→76; and 113→69; 127→66 amu, respectively. The parameters for the ion monitoring were as follows: spray voltage, 3.5 kV; sheath gas, 50; Aux gas, 10; sweep gas, 1; CE, 16.9 V for fluoromethylcholine, 26.7 V for fluoromethylbetaine, 30.3 V for d₉-choline, and 27.0 V for d₉-betaine; RF lens 47 for fluoromethylcholine, 58 for fluoromethylbetaine, 53 for d₉-choline, and 60 for d₉-betaine. Nitrogen (99.95% purity) was used as the source and argon (99.95 purity) was used as collision gas. Various concentrations of fluoromethylcholine and fluoromethylbetaine standards undergoing the same procedure were used to prepare the calibration curves for quantification of fluoromethylcholine and fluoromethylbetaine, respectively.

Data are shown in Table 2.

TABLE 2
PK Data

| Cpd. | Dose | 24 h fecal recovery (%) | Cmax-plasma (μM) |
|---|---|---|---|
| No. | (mg/kg) | (FMC and metabolites) | (FMC and metabolites) |
| 1 | 100 | 17.5 | 18.3 |
| 2 | 50 | 44.8 | 13.1 |
| 3 | 100 | 15.7 | 3.0 |
| 4 | 100 | 45.3 | 3.6 |
| 5 | 100 | 17.5 | 10.9 |
| 6 | 100 | 65.1 | 11.0 |

1. A compound of formula (I): 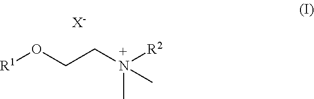
or a pharmaceutically acceptable salt thereof, wherein:
R¹ is selected from —C(O)ZR^{a} and —S(O)₂R^{b};
Z is selected from a bond, —O—, and —NH—;
R^{a} is selected from alkyl, alkenyl, alkynyl, aryl, and arylalkyl, each of which is optionally substituted with 1, 2, or 3 substituents independently selected from hydroxy, —COOH, alkyl, alkoxy, halo, haloalkyl, and a group 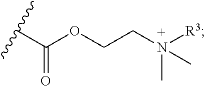
R^{b} is selected from alkyl, alkenyl, alkynyl, aryl, arylalkyl, and —NR^{c}R^{d};
R^{c} and R^{d} are each independently selected from hydrogen, alkyl, alkenyl, alkynyl, aryl, and arylalkyl;
R² and R³ are each independently selected from halomethyl and C₃-C₆ cycloalkyl; and
X⁻ is an anion.

2. The compound of claim 1, or a pharmaceutically acceptable salt thereof, wherein R¹ is —C(O)ZR^{a}.

3. The compound of claim 1 or claim 2, or a pharmaceutically acceptable salt thereof, wherein R^{a} is C₁-C₆ alkyl, which is unsubstituted or substituted with one —COOH group.

4. The compound of claim 1 or claim 2, or a pharmaceutically acceptable salt thereof, wherein R^{a} is aryl, which is unsubstituted or substituted with one substituent selected from —OH and a group

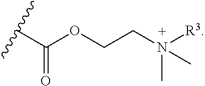

5. The compound of claim 4, or a pharmaceutically acceptable salt thereof, wherein R^{a} is phenyl substituted with one substituent selected from —OH and a group

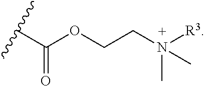

6. The compound of any one of claims 1-5, or a pharmaceutically acceptable salt thereof, wherein Z is a bond.

7. The compound of any one of claims 1-6, or a pharmaceutically acceptable salt thereof, wherein Z is O.

8. The compound of claim 1, or a pharmaceutically acceptable salt thereof, wherein R¹ is —S(O)₂R^{b}.

9. The compound of claim 8, or a pharmaceutically acceptable salt thereof, wherein R^{b} is NR^{c}R^{d}.

10. The compound of claim 9, or a pharmaceutically acceptable salt thereof, wherein R^{c} and R^{d} are each hydrogen.

11. The compound of any one of claims 1-10, or a pharmaceutically acceptable salt thereof, wherein R² is halomethyl.

12. The compound of any one of claims 1-11, or a pharmaceutically acceptable salt thereof, wherein R² is fluoromethyl.

13. The compound of any one of claims 1-12, or a pharmaceutically acceptable salt thereof, wherein R³ is halomethyl.

14. The compound of any one of claims 1-13, or a pharmaceutically acceptable salt thereof, wherein R³ is fluoromethyl.

15. The compound of any one of claims 1-14, wherein X⁻ is an anion selected from a halide and a carboxylate.

16. The compound of any one of claims 1-15, or a pharmaceutically acceptable salt thereof, wherein X⁻ is selected from chloride and bromide.

17. The compound of claim 1, wherein the compound is selected from: 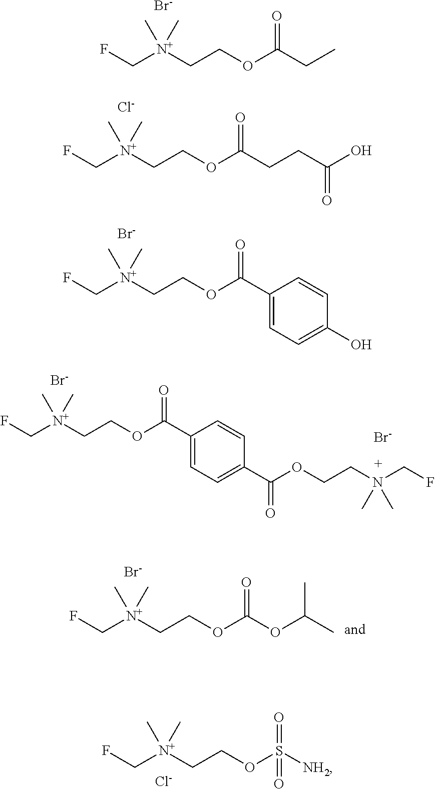
and pharmaceutically acceptable salts thereof.

18. A pharmaceutical composition comprising a compound of any one of claims 1-17, or a pharmaceutically acceptable salt thereof, and a pharmaceutically acceptable carrier.

19. A method of treating a condition associated with conversion of choline to trimethylamine in a subject in need thereof, comprising administering to the subject an effective amount of a compound of any one of claims 1-17, or a pharmaceutically acceptable salt thereof.

20. The method of claim 19, wherein the condition is selected from a cardiovascular disease, trimethylaminuria, reduced or impaired kidney function, kidney disease, diabetes mellitus, and obesity.

21. The method of claim 20, wherein the condition is a cardiovascular disease selected from acute coronary syndrome, angina, arrhythmia, arterial aneurysm, atherosclerosis, cardiomyopathy, congestive heart failure, coronary artery disease, carotid artery disease, endocarditis, coronary thrombosis, myocardial infarction, high blood pressure/hypertension, hypercholesterolemia/hyperlipidemia, peripheral artery disease, and stroke.

22. The method of claim 21, wherein the cardiovascular disease is due to oral biofilm formation and/or periodontal disease.

23. The method of claim 20, wherein the condition is a kidney disease selected from chronic kidney disease and end-stage renal disease.

24. The method of claim 19, wherein the condition is adverse ventricular remodeling, ventricular systolic dysfunction, ventricular diastolic dysfunction, cardiac dysfunction, or ventricular arrhythmia.

25. A method of improving or maintaining cardiovascular health in a subject in need thereof, comprising administering to the subject an effective amount of a compound of any one of claims 1-17, or a pharmaceutically acceptable salt thereof.

26. A method of inhibiting conversion of choline to trimethylamine in a subject in need thereof, comprising administering to the subject an effective amount of a compound of any one of claims 1-17, or a pharmaceutically acceptable salt thereof.

27. The method of claim 26, wherein the subject has an elevated level of trimethylamine N-oxide (TMAO) in blood, plasma, serum, urine, or any combination thereof.

28. A method of reducing a trimethylamine N-oxide level in a subject in need thereof, comprising administering to the subject an effective amount of a compound of any one of claims 1-17, or a pharmaceutically acceptable salt thereof.

29. The method of claim 28, wherein the subject has an elevated level of trimethylamine N-oxide (TMAO) in blood, plasma, serum, urine, or any combination thereof.

30. The method of any one of claims 19-29, further comprising administering a second therapeutic agent to the subject.

31. The method of claim 30, wherein the second therapeutic agent is selected from Omega 3 oil, salicylic acid, dimethylbutanol, garlic oil, olive oil, krill oil, Co enzyme Q-10, a probiotic, a prebiotic, dietary fiber, psyllium husk, bismuth salts, phytosterols, grape seed oil, green tea extract, vitamin D, antioxidants, turmeric, curcumin, and resveratrol.
